(19) United States
(12) Patent Application Publication
(10) Pub. No.: US 2007/0099917 A1
(43) Pub. Date: May 3, 2007

(54) Novel inhibitors and methods for their preparation

(75) Inventors: Edouard Collins Nice, Docklands (AU); Antony Wilks Burgess, Camberwell (AU); Keith Geoffrey Watson, Surrey Hills (AU)

Correspondence Address: SCULLY SCOTT MURPHY & PRESSER, PC, 400 GARDEN CITY PLAZA, SUITE 300, GARDEN CITY, NY, 11530, US

(73) Assignee: THE WALTER AND ELIZA HALL INSTITUTE OF MEDICAL RESEARCH, Parkville (AU)

(21) Appl. No.: 11/544,166

(22) Filed: Oct. 6, 2006

Related U.S. Application Data
(60) Provisional application No. 60/728,458, filed on Oct. 20, 2005.

(51) Int. Cl. A61K 31/5377; A61K 31/517; C07D 413/02; C07D 403/02; C07D 239/94

(52) U.S. Cl. 514/234.2; 514/266.4; 544/293; 544/120; 514/266.22; 544/284

(57) ABSTRACT

The present invention relates to novel tyrosine kinase (TK) inhibitors in the form of prodrugs.

### FIELD OF THE INVENTION

The present invention relates generally to antiproliferative agents and methods for their use and preparation. In particular the present invention relates to novel tyrosine kinase (TK) inhibitors in the form of prodrugs, methods for the preparation of these prodrugs and methods for using them in the treatment or prevention of proliferative disorders such as cancer.

### BACKGROUND OF THE INVENTION

Proliferative disorders are major life-threatening diseases and have been intensively investigated for decades. In particular, cancer now is the second leading cause of death in the United States, and over 500,000 people die annually from this proliferative disorder. All of the various cell types of the body can be transformed into benign or malignant tumour cells. Transformation of normal cells into cancer cells is a complex process and thus far is not fully understood. The current treatments available for cancer patients consist of one or a combination of surgery, radiation, and chemotherapy. While chemotherapy can be used to treat all types of cancer, surgery and radiation therapy are limited to particular cancers at certain sites of the body. There are a number of anticancer drugs widely used clinically. Many of these act by inhibiting DNA synthesis and are generally toxic to all cells and especially rapidly dividing cells such as tumour cells.

It is known that a cell may become cancerous by virtue of the transformation of a portion of its DNA into an oncogene. Several such oncogenes give rise to the production of peptides which are receptors for growth factors. It is the growth factor receptor complex which subsequently leads to an increase in cell proliferation. It is known, for example, that several oncogenes encode tyrosine kinase enzymes and that certain growth factor receptors are also tyrosine kinase enzymes.

Receptor tyrosine kinases have been recognised to be important in the transmission of biochemical signals which initiate cell replication. They are typically large enzymes which span the cell membrane and possess an extracellular binding domain for growth factors such as epidermal growth factor (EGF) and an intracellular portion which functions as a kinase to phosphorylate tyrosine amino acids in proteins and hence to influence cell proliferation. Various classes of receptor tyrosine kinases are known based on families of growth factors which bind to different receptor tyrosine kinases. The classification includes Class I receptor tyrosine kinases such as the EGF family of receptor tyrosine kinases which are frequently present in common human cancers such as breast cancer, non-small cell lung cancers (NSCLCs) including adenocarcinomas and squamous cell cancer of the lung, bladder cancer, oesophageal cancer, gastrointestinal cancer such as colon, rectal or stomach cancer, cancer of the prostate, leukaemia and ovarian, bronchial or pancreatic cancer. As further human tumour tissues are tested for the EGF family of receptor tyrosine kinases it is expected that its widespread prevalence will be established in further cancers such as thyroid and uterine cancer. It is also known that EGF type tyrosine kinase activity is rarely detected in normal cells whereas it is more frequently detectable in malignant cells. It has been shown recently that EGF receptors which possess tyrosine kinase activity are overexpressed in many human cancers such as brain, lung squamous cell, bladder, gastric, colorectal, breast, head and neck, oesophageal, gynaecological and thyroid tumours.

Accordingly it has been recognised that a selective inhibitor of receptor tyrosine kinases should be of value as a selective inhibitor of the growth of mammalian cancer cells. In particular the Class I receptor tyrosine kinase inhibitors are predicted to be useful in the treatment of a variety of human cancers. The use of inhibitors of EGF type receptor tyrosine kinases has also been postulated in the treatment of non-malignant proliferative disorders such as psoriasis, benign prostatic hypertrophy (BPH), atherosclerosis and restenosis.

The tyrphostin, 4-(3-chloroanilino)-6,7-dimethoxyquinazoline (also known as “AG1478”) (1), which is a competitive inhibitor of the ATP binding site in the kinase domain, is a highly potent and specific small molecule inhibitor of EGF type receptor tyrosine kinase and is a potential new anti-cancer agent. Unfortunately AG1478 is very hydrophobic and shows poor aqueous solubility, particularly at physiological salt concentrations. This in effect leads to poor bioavailability which is characterised by low circulating blood levels upon oral administration. This is not desirable, especially for prolonged therapeutical treatments.

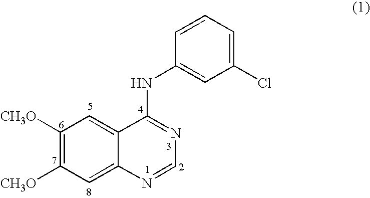

To improve the activity and/or pharmokinetic properties of AG1478 and related compounds, modifications of the core structure are often explored. For instance Bridges, et al, J. Med. Chem., 1996, 39, 267-276 reports on the structure activity relationship of various analogues of 4-anilino quinazolines including 6 and/or 7-amino substituted derivatives. Also, further derivatization may be carried out to introduce water-soluble moieties such as a dialkylamino group.

The pharmokinetic properties of many compounds which display poor oral bioavailability may be improved with the addition of prodrug substituents. One of the many available prodrug substituents is the 9-fluorenylmethoxycarbonyl-2-sulfonic acid or FMS moiety. The FMS moiety has been reported to increase the bioavailability of certain peptides and aminoglycosides (see for instance, Fridkin M., et al, J. Med. Chem., 2002, 45, 4264-4270). While the use of such prodrugs may appear common in improving the pharmacokinetic profile of water-insoluble drugs, it is well known that while one substituent may be successfully employed with a particular class of compounds, the same approach on a different class of compounds may be ineffective. The problem of finding a compatible prodrug substituent is compounded by the fact that there is a large choice of potential prodrug candidates which are structurally diverse and accordingly confer varying pharmokinetic properties.

Thus, seemingly minor modifications of a particular compound in order to accommodate a prodrug substituent or water-soluble moiety may cause the compound to become less effective or ineffective, or indeed lead to complications in the synthetic methodologies used to link the prodrug substituent to the active compound. As such while prodrug modification may often serve to enhance drug absorption and/or drug delivery into cells, the choice of prodrug substituent to achieve such an improved affect can often be unpredictable.

It has now been found that a novel class of prodrugs can be prepared, based upon the 4-anilino quinazoline structure of AG1478, which exhibits improved or useful drug absorption and drug delivery properties.

### SUMMARY OF THE INVENTION

The present invention provides a compound of formula (I) 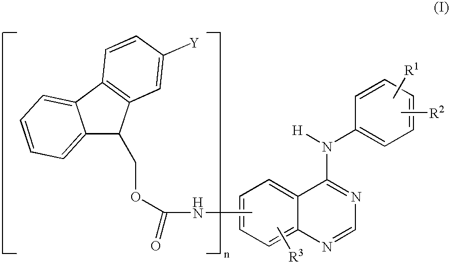
R¹ and R² are independently selected from hydrogen, hydroxy, halogen, nitro, cyano, optionally substituted alkyl, optionally substituted alkenyl, optionally substituted alkynyl, optionally substituted alkoxy, optionally substituted aryloxy, optionally substituted arylalkyloxy and optionally substituted heteroarylthio;

R³ is selected from hydrogen, hydroxy, halogen, nitro, cyano, carboxy, optionally substituted alkyl, optionally substituted alkoxy, aminoacyl, optionally substituted heteroaryl, and NR⁴R⁵ wherein R⁴ and R⁵ are independently selected from hydrogen, alkyl, cycloalkyl, aryl, —C(O)optionally substituted alkenyl, —C(O)optionally substituted alkyl and —C(O)optionally substituted alkynyl, or where R⁴ and R⁵ together with the N atom represent a 5, 6 or 7 membered nitrogen containing heterocycle;

Y is selected from hydrogen or sulfonic acid (SO₃H); and

n is either 1 or 2.

The present invention also provides a method for preparing a compound of formula (I), comprising contacting a compound of formula (II): 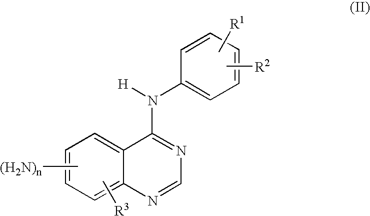
(wherein R¹-R³ and n are as defined above), with 9-fluorenylmethoxycarbonylchloride (FM-Cl), 9-fluorenylmethoxycarbonylchloride-2-sulfonic acid (FMS-Cl) or 9-fluorenylmethoxycarbonyloxy succinimide (FMOC-OSuc), for a time and under conditions sufficient to form said compound of formula (I), and optionally performing a chemical modification.

The present invention also provides a method for the treatment or prevention of proliferative disorders including the step of administering to a subject a therapeutically effective amount of a compound of formula (I).

### BRIEF DESCRIPTION OF FIGURES

FIG. 1. Fluorescence titration of the mesylate salt of AG1478 with NSA. Plot of the mesylate salt of AG1478 fluorescence as a function of added NSA (as equivalent HSA concentration). (10 μM of the mesylate salt of AG1478 in distilled water). Symbols (data). The solid line represents the fit to a 1:1 interaction with a K_{d} of 190 μM. (inset:plot with log scale on the concentration axis).

FIG. 2. Fluorescence titration of 6-amino-4-(3-chlorophenylamino)quinazoline. Plot of fluorescence as a function of added NSA (as equivalent HSA concentration). (10 μM 6-amino-4-(3-chlorophenylamino)quinazoline in distilled water). Symbols (data). The solid line represents a fit to a 1:1 interaction with a K_{d} of 174 μM.

FIG. 3. Fluorescence titration of FMS-6-amino-4-(3-chlorophenylamino)quinazoline with NSA. Plot of fluorescence as a function of added NSA (as equivalent HSA concentration). (0.1 μM FMS-6-amino-4-(3-chlorophenylamino)quinazoline in distilled water). Symbols (data). The solid line represents a fit to a 1:1 interaction with a K_{d} of 0.1 μM.

FIG. 4. Reverse Phase HPLC trace of FMS-6-amino-4-(3-chlorophenylamino)quinazoline and 6-amino-4-(3-chlorophenylamino)quinazoline. Plot of absorbance (mAU) as a function of time (min).

FIG. 5. Reverse Phase HPLC trace of FMS-6-amino-4-(3-chlorophenylamino)quinazoline and 6-amino-4-(3-chlorophenylamino)quinazoline in mouse plasma after 0 hrs. Plot of absorbance (mAU) as a function of time (min).

FIG. 6. Reverse Phase HPLC trace of FMS-6-amino-4-(3-chlorophenylamino)quinazoline and 6-amino-4-(3-chlorophenylamino)quinazoline in mouse plasma after 5 hrs. Plot of absorbance (mAU) as a function of time (min).

FIG. 7. Reverse Phase HPLC trace of FMS-6-amino-4-(3-chlorophenylamino)quinazoline and 6-amino-4-(3-chlorophenylamino)quinazoline in mouse plasma after 22 hrs. Plot of absorbance (mAU) as a function of time (min).

### DESCRIPTION OF THE PREFERRED EMBODIMENTS

The term “alkyl” as used herein refers to monovalent alkyl groups which may be straight chained or branched and preferably have from 1 to 10 carbon atoms, more preferably 1 to 6 carbon atoms and most preferably 1 to 4 carbon atoms. Examples of such alkyl groups include methyl, ethyl, n-propyl, iso-propyl, n-butyl, iso-butyl, and the like.

The term “alkylene” as used herein refers to divalent alkyl groups preferably having from 1 to 10 carbon atoms, more preferably 1 to 6 carbon atoms, and most preferably 1 to 4 carbon atoms. Examples of such alkylene groups include methylene (—CH₂—), ethylene (—CH₂CH₂—), and the propylene isomers (e.g., —CH₂CH₂CH₂— and —CH(CH₃)CH₂—), and the like.

The term “aryl” as used herein refers to an unsaturated aromatic carbocyclic group having a single ring (eg., phenyl) or multiple condensed rings (eg., naphthyl or anthryl), preferably having from 6 to 14 carbon atoms. Examples of aryl groups include phenyl, naphthyl and the like.

The term “aryloxy” as used herein refers to the group aryl-O— wherein the aryl group is as described above.

The term “arylalkyl” as used herein refers to -alkylene-aryl groups preferably having from 1 to 10 carbon atoms in the alkylene moiety and from 6 to 10 carbon atoms in the aryl moiety, more preferably 1 to 6 carbon atoms in the alkylene moiety and most preferably 1 to 4 carbon atoms in the alkylene moiety. Such arylalkyl groups are exemplified by benzyl, phenethyl and the like.

The term “arylalkoxy” as used herein refers to the group arylalkyl-O— wherein the arylalkyl group is as described above. Such arylalkoxy groups are exemplified by benzyloxy and the like.

The term “alkoxy” as used herein refers to the group alkyl-O— where the alkyl group is as described above. Examples include, methoxy, ethoxy, n-propoxy, iso-propoxy, n-butoxy, tert-butoxy, sec-butoxy, 1,2-dimethylbutoxy, and the like.

The term “alkenyl” as used herein refers to a monovalent alkenyl groups which may be straight chained or branched and preferably have from 2 to 10 carbon atoms, more preferably 2 to 6 carbon atoms and most preferably 2 to 4 carbon atoms and have at least 1 and preferably from 1-2, carbon to carbon, double bonds. Examples include ethenyl (—CH═CH₂), n-propenyl (—CH₂CH═CH₂), iso-propenyl (—C(CH₃)═CH₂), but-2-enyl (—CH₂CH═CHCH₃), and the like.

The term “alkynyl” as used herein refers to monovalent alkynyl groups which may be straight chained or branched and preferably have from 2 to 10 carbon atoms, more preferably 2 to 6 carbon atoms and most preferably 2 to 4 carbon atoms and have at least 1, and preferably from 1-2, carbon to carbon, triple bonds. Examples of alkynyl groups include ethynyl (—C≡CH), propargyl (—CH₂C≡-CH), pent-2-ynyl (—CH₂C≡CCH₂—CH₃), and the like.

The term “aminoacyl” as used herein refers to the group —C(O)NR″R″ where each R″ is independently hydrogen, alkyl, cycloalkyl and aryl, and where each of alkyl, aryl, and cycloalkyl, is as described herein.

The term “cycloalkyl” as used herein refers to cyclic alkyl groups having a single cyclic ring or multiple condensed rings, preferably incorporating 3 to 8 carbon atoms. Such cycloalkyl groups include, by way of example, single ring structures such as cyclopropyl, cyclobutyl, cyclopentyl, cyclohexyl, cyclooctyl, and the like, or multiple ring structures such as adamantanyl, and the like.

The term “halo” or “halogen” as used herein refers to fluoro, chloro, bromo and iodo.

“Heteroaryl” refers to a monovalent aromatic carbocyclic group, preferably of from 2 to 10 carbon atoms and 1 to 4 heteroatoms selected from oxygen, nitrogen and sulfur within the ring. The most preferred heteroatoms are nitrogen, oxygen, and sulfur. Such heteroaryl groups can have a single ring (e.g., pyridyl, pyrrolyl, imididazolyl, thienyl, or furanyl) or multiple condensed ring s (e.g., indolizinyl or benzothienyl).

“Heteroarylthio” as used herein refers to the group —S-heteroaryl wherein the heteroaryl group is as described above.

“Heterocyclyl” as used herein refers to a monovalent saturated or unsaturated group having a single ring or multiple condensed rings, preferably from 1 to 8 carbon atoms and from 1 to 4 hetero atoms selected from nitrogen, sulfur, oxygen, selenium or phosphorous within the ring. The most preferred heteroatoms are nitrogen and oxygen. Examples of heterocyclyl groups include morpholinyl, piperidinyl and piperazinyl.

In this specification the term “optionally substituted” is taken to mean that a group may or may not be further substituted or fused (so as to form a condensed polycyclic group) with one or more groups selected from hydroxyl, acyl, alkyl, alkoxy, alkenyl, alkenyloxy, alkynyl, alkynyloxcy, amino, aminoacyl, thio, arylalkyl, arylalkoxy, aryl, aryloxy, carboxyl, cycloalkyl, cyano, halogen, nitro, sulphate, phosphate, heterocyclyl, trihalomethyl, trialkylsilyl, 2-(2-methanesulphonyl-ethylamino)methyl, morpholinomethyl, 3-ethoxy-N-morpholinomethyl, and methyldisulfanyl.

In preferred embodiments one or more of the following definitions apply.

R¹ and R² are preferably independently selected from the following groups:
hydrogen;
hydroxy;
halogen; more preferably fluoro, chloro, and bromo;
nitro;
cyano;
C₁-C₄ alkyl, more preferably methyl and ethyl;
C₁-C₄ halo substituted alkyl, more preferably 1-chloroethyl, trifluoromethyl;
C₂-C₄ alkenyl, more preferably vinyl and allyl;
substituted C₂-C₄ alkenyl, preferably, nitrovinyl, cyano vinyl, or trifluorovinyl;
C₂-C₄ alkynyl, preferably 1-propynyl or ethynyl;
C₁-C₄ alkoxy, more preferably methoxy and ethoxy;
C₁-C₄ halo substituted alkoxy, more preferably 1-chloroethyloxy;
aryloxy, more preferably phenoxy;
substituted aryloxy, more preferably halogen substituted aryloxy;
arylalkyloxy, more preferably benzyloxy;
substituted arylalkoxy, more preferably halogen substituted arylalkyloxy; and
substituted heteroarylthio, more preferably substituted thioimidazole, especially 1-methylimidazol-2-ylthio.

Most preferred substituents for R¹ and R² are independently selected from hydrogen, fluoro, chloro, bromo, methyl, trifluoromethyl, vinyl, ethynyl, phenoxy, benzyloxy, 3-F-benzyloxy and 1-methylimidazol-2-ylthio.

It is even more preferred that where one of R¹ and R² is other than H it is located at the 3-position, and where both R¹ and R² are other than H, R¹ and R² are located at the 3- and 4-positions.

Especially preferred substitution patterns for R¹, R² include: 3-Cl, 4-H; 3-Br, 4-H; 3-methyl, 4-H; 3-Cl, 4-F; 3-ethynyl, 4-H; 3-H, 4-H; 3-Cl, 4-OCH₂(3-FC₆H₄); 3-H, 4-OC₆H₅; 3-Br, 4-(1-methylimidazol-2-ylthio); 3-H, 4-OCH₂C₆H₅.

R³ is preferably selected from the following groups:
hydrogen;
hydroxy;
halogen, more preferably fluoro, chloro, and bromo;
nitro;
cyano;
C₁-C₄ alkyl, more preferably methyl and ethyl;
C₁-C₄ halo substituted alkyl, more preferably 1-chloroethyl;
C₁-C₄ alkoxy, more preferably methoxy and ethoxy;
C₁-C₄ substituted alkoxy, more preferably halo substituted alkoxy such as 1-chloroethoxy, as well as methoxyethoxy, 2-(N-morpholinyl) ethoxy, 3-(N-morpholinyl) propoxy;
aminoacyl, preferably carbamoyl, N-(C₁-C₄)alkyl carbamoyl, N,N′-di-(C₁-C₄)alkyl carbamoyl;
NR⁴R⁵, preferably C₁-C₄ mono or dialkylamino, more preferably N-methylamino, N,N′-dimethylamino, —NHC(O)CH₂SSCH₃, —NHC(O)CHCHCH₂(3-ethoxy-N-morpholinyl), acrylamide, butynamide, propanamide, or R⁴ and R⁵ together with the N atom represent morpholinyl or piperidinyl;
optionally substituted heteroaryl, preferably imidazolyl, 2-((2-methanesulphonyl-ethylamino)methyl)-furan-2-yl and 5-morpholinomethylthien-3-yl.

Most preferred substituents for R³ are selected from hydrogen, hydroxy, fluoro, chloro, methoxy, ethoxy, methyl, methoxyethoxy, 2-(N-morpholinyl)ethoxy, 3-(N-morpholinyl)propoxyl, N-methylamino, N,N′-dimethylamino, acrylamidyl, butynamidyl, propanamidyl, 1-imidazolyl, —NHC(O)CH₂SSCH₃, —NHC(O)CHCHCH₂(3-ethoxy-N-morpholinyl), morpholinyl, piperidinyl, 2-((2-methanesulphonyl-ethylamino) methyl)-furan-2-yl and 5-morpholinomethylthien-3-yl.

The prodrug moieties are preferably located at the 7- and/or 6-positions of the quinazoline ring, although n is preferably 1.

Preferably when n is 1 the prodrug moiety is located at the 6- or 7-position of the quinazoline ring and R³ is located at the vacant 6- or 7-position adjacent to the prodrug moiety. Most preferably the prodrug moiety is located at the 6-position of the quinazoline ring and the R³ group is located at the 7-position of the quinazoline ring.

Preferably when n is 2 both Y are the same. In this embodiment it is preferred that R³ is H, F, or Cl and most preferably H.

In compounds of formula (I) where a chiral centre is present, the invention encompasses enantiomers or stereoisomers and mixtures thereof, such as enantiomerically enriched mixtures. It will be appreciated that the quinazoline system in the present compounds may exist as rapidly interconvertible mixtures of isomers. Isomerism of this kind is known in the art as “tautomerism”. Individual isomers are called tautomers. Where tautomerism is possible the present invention encompasses all possible tautomers of the compounds of formula (I).

The compounds of the present invention may be prepared and administered to a subject as pharmaceutically acceptable salts. Suitable pharmaceutically acceptable salts include, but are not limited to salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quartemised with such agents as lower alkyl halide, such as methyl, ethyl, propyl, and butyl chlorides, bromides and iodides; dialkyl sulfates like dimethyl and diethyl sulfate; and others.

The compounds of formula (I) have a 4-anilinoquinazoline framework with one or two amino groups substituted on the phenyl ring which is fused to the pyrimidine which forms the quinazoline ring system. The amino group(s) is/are further substituted with a N-[(2-Y-substituted)-9-fluorenylmethoxycarbonyl] moiety, where Y is as defined above.

The compounds of formula (I) of the present invention can be prepared using processes known to be applicable to the preparation of 4-anilinoquinazolines. It is generally preferred that the N-[(2-optionally substituted)-9-fluorenylmethoxycarbonyl] prodrug moiety or moieties is/are added after the assembly of the 4-anilinoquinazoline. Accordingly a key synthetic intermediate of the compounds of the present invention is represented by general formula (II), wherein R¹-R³ and n are as defined above.

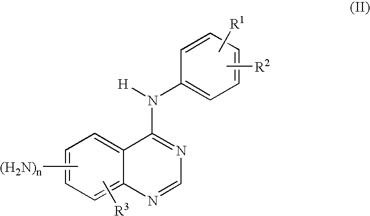

For instance amino substituted 4-anilinoquinazolines of formula (II) may be prepared by reacting substituted anilines with quinazolines which bear a suitable leaving group at the 4-position. Thus, the compounds of formula (II) may be prepared by reacting a compound of formula (III): 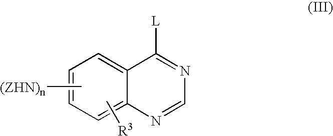
(wherein R³ and n are as defined above, L is a leaving group and Z is H or a nitrogen protecting group), with a compound of formula (IV): 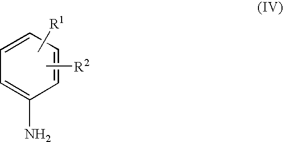
(wherein R¹ and R² are as defined above), and subsequently removing the nitrogen protecting group Z, if present.

Preferred leaving groups as L include halogen, C₁-C₄ alkoxy, aryloxy or sulphonyloxy groups such as methanesulphonyloxy or toluene-4-sulphonyloxy.

Suitable protecting groups (Z) for NH₂ are known in the art including an acyl group. Other suitable protecting groups are described in Greene, T. W., et al in Protecting Groups in Organic Synthesis, 3^{rd} edition, John Wily & Sons, Inc. (1999), which is incorporated herein by reference.

The reaction described above is preferably conducted in the presence of either an acid or base catalyst. Suitable acids include, for example, inorganic acids such as HCl. Suitable bases include, for example, organic bases such as pyridine, triethylamine, Hünigs base, morpholine, N-methylmorpholine, DBU; an alkali metal or alkaline earth metal hydroxide or carbonate, for example sodium hydroxide, potassium carbonate; alkali metal hydrides, for example sodium hydrides and the like.

The reaction is preferably conducted in the presence of a solvent. Suitable solvents include polar solvents such as ethanol, 2-propanol, or polar aprotic solvents such as DMF, N,N′-dimethyl acetamide, N-methylpyrrolidine-2-one or DMSO, or halogenated solvents such as dichloromethane, carbon tetrachloride and the like.

The reaction is stirred in a round bottom flask under atmospheric conditions or preferably under an inert environment, for instance, under nitrogen or argon. Preferably the reaction is conducted at elevated or solvent refluxing temperatures between the range of 20-80° C.

When the reaction is conducted using as a reactant a compound of formula (III) which bears a protected NH₂ group (that is, ZHN), the protecting group may be removed by any of the conventional methods described in Green, T. W., et al, Protecting Groups in Organic Synthesis, which is referred to above.

The compounds of general formula (II) may be obtained from the above process as either the salt (for instance, with the acid of formula H-L) or in free form. When it is preferred to liberate the free base from the salt form, a base may be added according to conventional methods.

Furthermore, where any one of R¹, R² and R³ are reactive groups such as hydroxy groups they may be protected prior to being subjected to the conditions described above. Suitable hydroxyl protecting groups are known in the art, for example, silyl ethers. Such protecting groups are again outlined in Green, T. W., et al, “Protective Groups in Organic Synthesis”, as referred to above.

An alternate route to compounds of formula (II) is illustrated below in Scheme 1. This methodology is derived from Tsou., H-R, et al, J. Med. Chem., 2001, 44, 2719-2734.

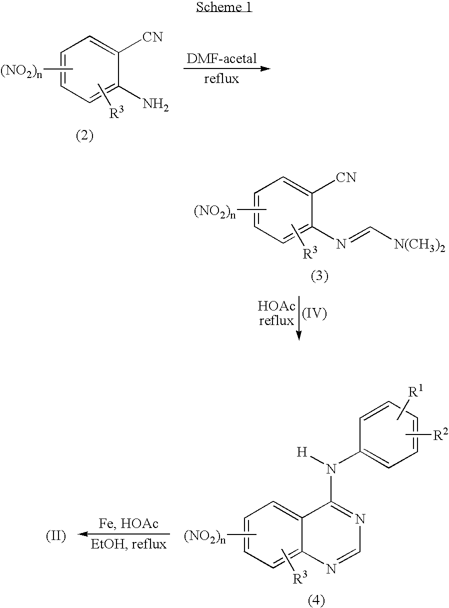

As illustrated in scheme 1 the synthesis allows for the preparation of the quinoxaline ring and the introduction of the 4-anilino group in a single step.

The synthetic procedure depicted in Scheme 1 above may commence from a commercially available nitro substituted anthranilonitrile (2), or by first preparing the desired nitro or dinitro substituted anthranilonitrile through nitration of commercially available anthranilonitrile (Aldrich Chemicals).

The starting anthranilonitrile (2) is converted into the corresponding formamidine (3) by refluxing with dimethylformamide dimethyl acetal.

Further refluxing of a solution of (3) with an aniline of formula (IV) produces the 4-anilino substituted quinoxaline (4). Reduction of the nitro group(s) with iron in an ethanol/acetic acid solution (under reflux) affords the desired compound of formula (II).

The preparation of variously substituted 4-anilinoquinazolines is also reported in the following published International applications: WO 95/23141 (Pfizer, Inc.), WO 96/16960 (Zeneca Limited), WO 97/30044 (Zeneca Limited), WO 96/15118 (Zeneca Limited), WO 99/09016 (American Cyanamid Co.) and WO 01/04111 (Glaxo Group Limited).

The compounds of formula (I) can be preferably prepared by reacting compounds of formula (II) with 9-fluorenylmethoxycarbonylchloride (FM-Cl) or 9-fluorenylmethoxycarbonylchloride-2-sulfonic acid (FMS-Cl). FMS-Cl can be prepared by the method of Merrifield and Bach, J. Org. Chem., 43 (1978) 4808-4816. FM-Cl is commercially available from Sigma-Aldrich Pty. Ltd (as FMOC-Cl). Alternatively, the compounds of the present invention may be prepared by reacting compounds of formula (II) with 9-fluorenylmethoxycarbonyloxysuccinimide (FMOC-OSuc) or 9-fluoroenylmethoxycarbonyl-N-hydroxysuccinimide-2-sulfonic acid (FMS-OSuc). FMOC-OSuc is commercially available from Sigma-Aldrich Pty. Ltd (as Fmoc-OSu), and FMS-OSuc may be prepared as reported by Fridkin et al, J. Med. Chem., 2002, 45, 4264.

The reaction is preferably conducted in a solvent, such as ethyl acetate, acetonitrile, acetone or dichloromethane and a base is also preferably added to neutralise the acid (HCl or HOSuc) formed during the reaction. Suitable neutralising bases include potassium acetate, potassium carbonate, tertiary amines such as triethylamine and Hünigs base as well as basic resins. Preferably when the reaction involves the use of FM-Cl or FMS-Cl, it is conducted in ethyl acetate with the addition of potassium acetate as the neutralising base. In this embodiment the base is preferably added in mole excess relative to the compound of formula (II). Preferably the FM-Cl, FMS-Cl, FMOC-OSuc or FMS-OSuc is added in a mole equivalent amount relative to the compound of formula (II).

The reaction is preferably gently stirred under inert conditions, for instance, under a nitrogen or argon atmosphere, or in a round bottom flask equipped with a drying tube. The progress of the reaction may be monitored by conventional techniques such as thin layer chromatography, HPLC, NMR or IR spectroscopy. Reaction times may vary depending upon the exact nature of the conditions and reactants but generally the reaction is observed to reach completion between 6-72 days and typically around 48 days with the use of FMS-Cl in ethyl acetate with added potassium acetate.

The product may be recovered from the reaction mixture with the addition of a portion of ice water and then shaking and filtering the resultant suspension. The desired product (compound of formula (I)), which is often solid, is then preferably washed with further portions of cold water and ethyl acetate.

Further purification of the product can be achieved by recrystallisation, silica column chromatography, or reverse phase HPLC.

The compounds of formula (I) prepared by this method may be converted to other compounds of formula (I) by optionally performing a chemical modification. Such modification may involve transformations through functionalisation or functional group interconversion or where a group is protected, a deprotection step. Accordingly, the chemical modification may include esterification, bromination, amination, nitration, diazotization and nucleophile replacement (e.g., with Hal⁻ or CN⁻) or deprotection.

The compounds of the present invention represent administrable prodrugs. The term “prodrug” specifically relates to the —[N-9-(2-optionally substituted) fluorenylmethoxycarbonyl] group(s), which are converted to —NH₂ group(s) in vivo. The in vivo conversion of the prodrug may be facilitated either by cellular enzymes such as lipases and esterases or by chemical cleavage such as in vivo hydrolysis. Although the compounds of formula (I) may not themselves be biologically active, they are readily hydrolysed to yield the biologically active form. A main advantage of the present compounds however is that they typically display extended half-lives with plasma and accordingly are predicted to greatly improve the bioavailability and pharmacokinetics of the active drug. Accordingly, the prodrugs of the present invention may act to enhance drug adsorption and/or drug delivery into cells.

Without wishing to be bound by theory it is believed that the extended half-life exhibited by the FMOC/FM/FMS prodrugs of the present invention may be a result of an endogenous association with albumin with a sufficient affinity to slow the rate of clearance (by glomeruler filtration) of the parent active compound.

The compounds of the present invention are useful for the inhibition of protein kinases and in particular, EGF type receptor tyrosine kinases.

Accordingly, the compounds of the present invention may be useful as therapeutics for the treatment of proliferative disorders, including, but not limited to, lung cancer; bladder cancer; liver cancer; prostate cancer; colon, rectal or stomach cancer; breast cancer; ovarian cancer; pancreatic cancer; melanoma, and leukemia; as well as non-malignant proliferative disorders such as psoriasis; benign prostatic hypertrophy; atherosclerosis and restenosis.

The compounds of the present invention may be therapeutically administered as a single drug, or alternatively may be administered in combination with one or more other active chemical entities to form a combination therapy. The other active chemical entities may be a small molecule, a polypeptide, or a polynucleotide.

The compounds of the present invention are preferably administered as a pharmaceutically acceptable composition. The pharmaceutical composition of the present invention comprises a therapeutically effective amount of at least one of the compounds represented by formula (I) or pharmaceutically acceptable salt thereof as active ingredients.

The compositions include those suitable for oral, topical, intravenous, subcutaneous, nasal, ocular, pulmonary, and rectal administration. Preferably, the administration route is oral. The compounds of the invention may be administered to mammalian individuals, including humans, as therapeutic agents.

The term “treatment” as used in the phrase “for the treatment or prevention of proliferative disorders” is intended to include any means of treating a proliferative disease in a mammal, including (1) inhibiting the disease, that is, arresting the development or progression of clinical symptoms, and/or (2) relieving the disease, i.e., causing regression of clinical symptoms.

The term “prevention” as used in the phrase “for the treatment or prevention of proliferative disorders” is intended to include any means of preventing the onset of the disease state, i.e., avoiding any clinical symptoms of the disease.

A “therapeutically effective amount” of a compound of the invention refers to an amount which is effective, upon single or multiple dose administration to the patient, in controlling the growth of e.g., a tumour or in prolonging the survivability of the patient beyond that expected in the absence of such treatment. As used herein, “controlling the growth” refers to slowing, interrupting, arresting or stopping the proliferative transformation of cells and does not necessarily indicate a total elimination of e.g., the tumour.

Dosing may occur at intervals of minutes, hours, days, weeks, months or years or continuously over any one of these periods. Suitable dosages lie within the range of about 0.1 ng per kg of body weight to 1 g per kg of body weight per dosage. The dosage is preferably in the range of 1 μg to 1 g per kg of body weight per dosage, such as is in the range of 1 mg to 1 g per kg of body weight per dosage. In one embodiment, the dosage is in the range of 1 mg to 500 mg per kg of body weight per dosage. In another embodiment, the dosage is in the range of 1 mg to 250 mg per kg of body weight per dosage. In yet another preferred embodiment, the dosage is in the range of 1 mg to 100 mg per kg of body weight per dosage, such as up to 50 mg per body weight per dosage.

Suitable dosage amounts and dosing regimens can be determined by an appropriately skilled physician and may depend on the particular condition being treated, the severity of the condition as well as the general age, health and weight of the subject.

Accordingly, the present invention includes pharmaceutical compositions comprising, as an active ingredient, at least one of the compounds of the invention in association with a pharmaceutical carrier.

The compounds of this invention can be administered by oral, parenteral (intramuscular, intraperitoneal, intravenous (IV) or subcutaneous injection), topical, transdermal (either passively or using iontophoresis or electroporation), transmucosal (e.g., nasal, vaginal, rectal, or sublingual) or pulmonary (e.g., via dry powder inhalation) routes of administration or using bioerodible inserts and can be formulated in dosage forms appropriate for each route of administration.

Solid dosage forms for oral administration include capsules, tablets, pills, powders, and granules. In such solid dosage forms, the active compound is admixed with at least one inert pharmaceutically acceptable carrier such as sucrose, lactose, or starch. Such dosage forms can also comprise, as is normal practice, additional substances other than inert diluents, e.g., lubricating, agents such as magnesium stearate. In the case of capsules, tablets, and pills, the dosage forms may also comprise buffering agents. Tablets and pills can additionally be prepared with enteric coatings.

Liquid dosage forms for oral administration include pharmaceutically acceptable emulsions, solutions, suspensions, syrups, with the elixirs containing inert diluents commonly used in the art, such as water. Besides such inert diluents, compositions can also include adjuvants, such as wetting agents, emulsifying and suspending agents, and sweetening, flavouring, and perfuming agents.

Preparations according to this invention for parenteral administration include sterile aqueous or non-aqueous solutions, suspensions, or emulsions. Examples of non-aqueous solvents or vehicles are propylene glycol polyethylene glycol, vegetable oils, such as olive oil and corn oil, gelatine, and injectable organic esters such as ethyl oleate. Such dosage forms may also contain adjuvants such as preserving, wetting, emulsifying, and dispersing agents. They may be sterilised by, for example, filtration through a bacteria retaining filter, by incorporating sterilising agents into the compositions, by irradiating the compositions, or by heating the compositions. They can also be manufactured using sterile water, or some other sterile injectable medium, immediately before use.

Compositions for rectal or vaginal administration are preferably suppositories which may contain, in addition to the active substance, excipients such as cocoa butter or a suppository wax. Compositions for nasal or sublingual administration are also prepared with standard excipients well known in the art.

Topical formulations will generally comprise ointments, creams, lotions, gels or solutions. Ointments will contain a conventional ointment base selected from the four recognised classes: oleaginous bases; emulsifiable bases; emulsion bases; and water-soluble bases. Lotions are preparations to be applied to the skin or mucosal surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Creams, as known in the art, are viscous liquid or semisolid emulsions, either oil-in-water or water-in-oil. Topical formulations may also be in the form of a gel, i.e., a semisolid, suspension-type system, or in the form of a solution.

Finally, formulations of these drugs in dry powder form for delivery by a dry powder inhaler offer yet another means of administration. This overcomes many of the disadvantages of the oral and intravenous routes.

Certain embodiments of the invention will now be described with reference to the following examples which are intended for the purpose of illustration only and are not intended to limit the scope of the generality hereinbefore described.

### EXAMPLES

### Synthetic Procedures

### Example 1

### a) Synthesis of the Fluorenylmethoxycarbonyl-2-sulfonic acid (FMS) 6-aminoguinazoline Derivative

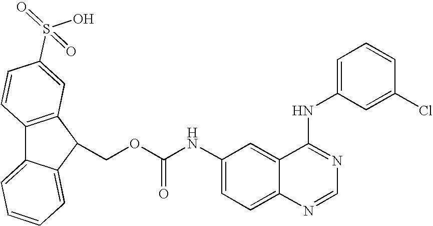

6-Amino-4-(3-chlorophenylamino)quinazoline was prepared from Tsou, H-R et al, J. Med. Chem 44 (2001) 2719-2734 and 9-Fluorenylmethoxycarbonylchloride-2-sulfonic acid (FMS-Cl) was prepared as reported by Merrifield and Bach, J. Org. Chem. 43 (1978) 4808-4816.

A suspension of 6-amino-4-(3-chlorophenylamino)quinazoline (270 mg, 1.0 mmol), 9-fluorenylmethoxycarbonylchloride2-sulfonic acid (340 mg, 1.0 mmol) and potassium acetate (150 mg, 1.5 mmol) in ethyl acetate (15 mL) was gently stirred at room temperature under a drying tube for 48 days. The reaction mixture was poured into a separating funnel and extra ethyl acetate (100 mL) was added and then ice water (100 mL). The mixture was shaken vigorously for several minutes and then the suspension was filtered to give a pale olive solid which was washed with further ice water (50 mL) and ethyl acetate (50 mL) and then the solid was dried in a vacuum oven to give the crude product as an olive-coloured solid (420 mg, 73%), ¹H NMR spectrum (d₆DMSO): δ 4.35 (m, 1H), 4.64 and 4.79 (each dd, 1H), 7.33-7.9 (complex m, 15H), 8.06 (s, 1H), 8.72 (s, 1H), 8.85 (s, 1H), 10.19 (s, 1H), 11.50 (bs, 1H); Mass spectrum (ESI) m/z 573 (M+H)⁺.

### b) Reversed Phase (RP) HPLC purification of the (FMS) 6-aminoquinazoline Derivative

i) The (FMS) 6-aminoquinazoline derivative from a) was separated from unreacted 6-amino-4-(3-chlorophenylamino)quinazoline by RP-HPLC. Separation was performed using an Agilent Model 1100 HPLC (Agilent Technologies, Victoria, Australia) equipped with a diode array detector and using a Brownlee Aquapore RP-300 (Octyl C8) 7 μm 100×2.1 mm column. Separation was achieved by gradient elution using a linear 40 min gradient between 10 mM ammonium acetate pH6.0 and 100% acetonitrile. The flow rate was 0.1 ml/minute and the column temperature was 25° C. Detection was at 330 nm. Fractions were recovered manually, allowance being made for the dead volume between the detector and trapping port. ESI-MS analysis of the purified material, which had a characteristic retention time of 23.9 min., gave the anticipated mass of 573.2 (M+H)⁺.
ii) The (FMS) 6-aminoquinazoline derivative from a) was separated from unreacted 6-amino-4-(3-chlorophenylamino)quinazoline by RP-HPLC. Separation was performed using an Zorbax SB-C18, 2.1 ×150 mm, 5μ column using a linear gradient of 10 mM ammonium acetate pH 6.0 to acetonitrile (5-95% buffer B) over 45 min., at a flow rate of 0.1 ml per minute and a column temperature of 45° C. Detection was at 345 nm. There was a major peak at 29 minutes and minor one at 31 minutes (see FIG. 4). A standard sample of 6-amino-4-(3-chlorophenylamino)-quinazoline had a retention time of 31 minutes. These conditions were used to make a purified AG1478-6-amino-FMS sample for further experiments.

### Example 2

### a) Synthesis of the Fluorenylmethoxycarbonyl Derivative of 6-amino-4-(3-chlorophenylamino)quinazoline

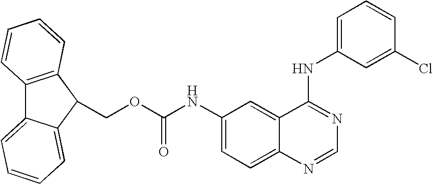

A solution of N-(9-fluorenylmethoxycarbonyloxy)succinimide (70 mg, 0.2 mmol) in dichloromethane (3 mL) was added at room temperature with stirring to a suspension of 6-amino-4-(3-chlorophenylamino)quinazoline (55 mg, 0.2 mmol) in dichloromethane (5 mL). The reaction was stirred at room temperature and was monitored by thin-layer chromatography (tlc). After 6 days tlc (silica/chloroform) showed, in addition to both starting materials, the presence of a new compound of intermediate polarity. The reaction mixture was poured into a separating funnel and washed with dilute sodium bicarbonate solution. The dichloromethane layer was separated and dried (MgSO₄) and then evaporated to give a solid residue (100 mg) which was immediately chromatographed on silica gel (7 g) using chloroform as eluent. The fractions were compared by tlc and those containing a compound of intermediate polarity (slower running than FMOC-OSuc) were combined. Evaporation of the solvent gave the product as a pale brown solid (20 mg, 19%). LC/MS analysis showed a single peak and mass spectrum (ESI) gave the expected peaks for a mono-chlorinated compound m/z 493 and 495 (M+H). ¹H NMR spectrum (CDCl₃+1 drop CD₃OD): δ 4.21 (t, 1H), 4.47 (d, 2H), 7.17-7.35 (m, 6H), 7.55-7.63 (m, 4H), 7.66-7.72 (m, 3H), 7.74 (t,1H), 7.87-7.96 (m, 2H), 8.45 (bs, 1H), 8.49 (s, 1H).

Biological Data

TABLE 1
Binding affinities (K_{d}) for normal serum albumin (NSA)

| | mesylate | 6-amino-4-(3- | FMS-6-amino-4-(3- |
|---|---|---|---|
| | salt of | chlorophenylamino) | chlorophenylamino) |
| Sample | AG1478 | quinazoline | quinazoline |
| K_{d} | 190 μM | 174 μM | 0.1 μM |

Standard Protocol

NSA (a 20% w/v human serum albumin preparation for intravenous administration from pooled human plasma prepared by ethanol fractionation and chromatography) was obtained from CSL Limited, Broadmeadows, Victoria, 3047, Australia.

The binding of the mesylate salt of AG1478 to NSA was determined by the increase in quantum yield of mesylate salt of AG1478 fluorescence. Fluorescence titrations were conducted with a SPEX Fluoro-Log Tau-2 Frequency-domain Spectrofluorometer operated in the steady-state mode at a temperature of 20° C. Mesylate salt of AG1478 was excited with vertically polarized emission from a Xenon lamp at a wavelength of 350 nm. Mesylate salt of AG1478 emission was collected at the magic angle polarization (55.7°) at 400 nm. Quartz cuvettes containing a fixed concentration of mesylate salt of AG1478 10 μM in distilled water), were titrated with variable amounts of NSA. Background scatter and background fluorescence were corrected for using blank samples containing only NSA. The fluorescence data was analysed to either a single-site or two-independent-sites models using non-linear least squares fitting (Microsoft Excel and Prism). For single binding, the emission intensity (y) as a finction of added NSA was fit to the function y=(A/2/D)*[C+X+D−SQRT((C+X+D)2-4XD)], where A=change in intensity at saturation, B=initial intensity, C=K_{d}, D=concentration of AG1478 (fixed) and X=concentration of NSA. The change in intensity at saturation was either used as a fitting parameter (in cases where obvious saturation was observes) or extrapolated using a double reciprocal plot.

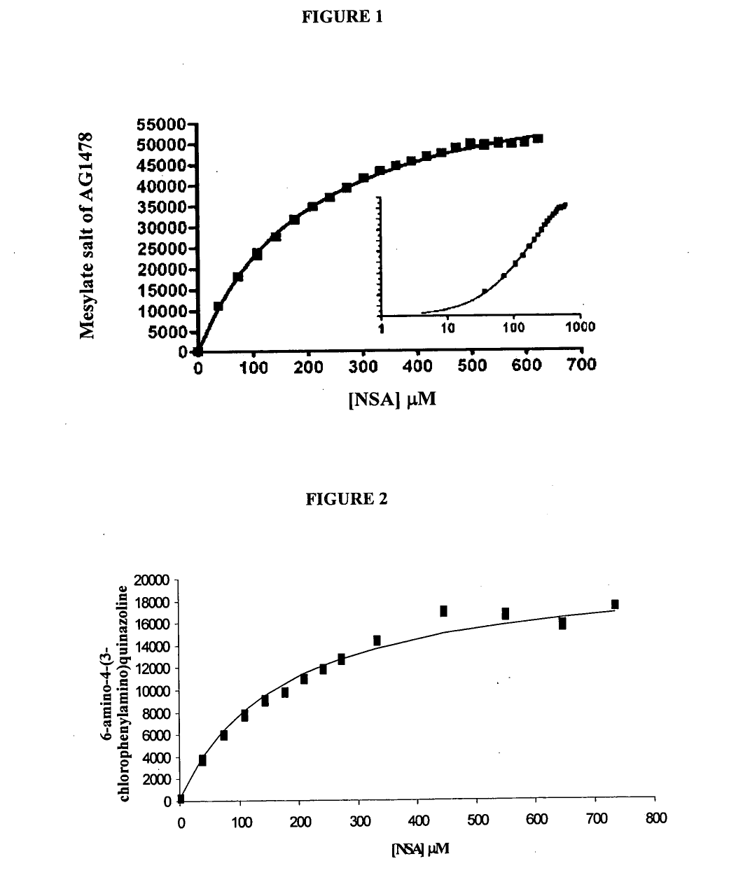

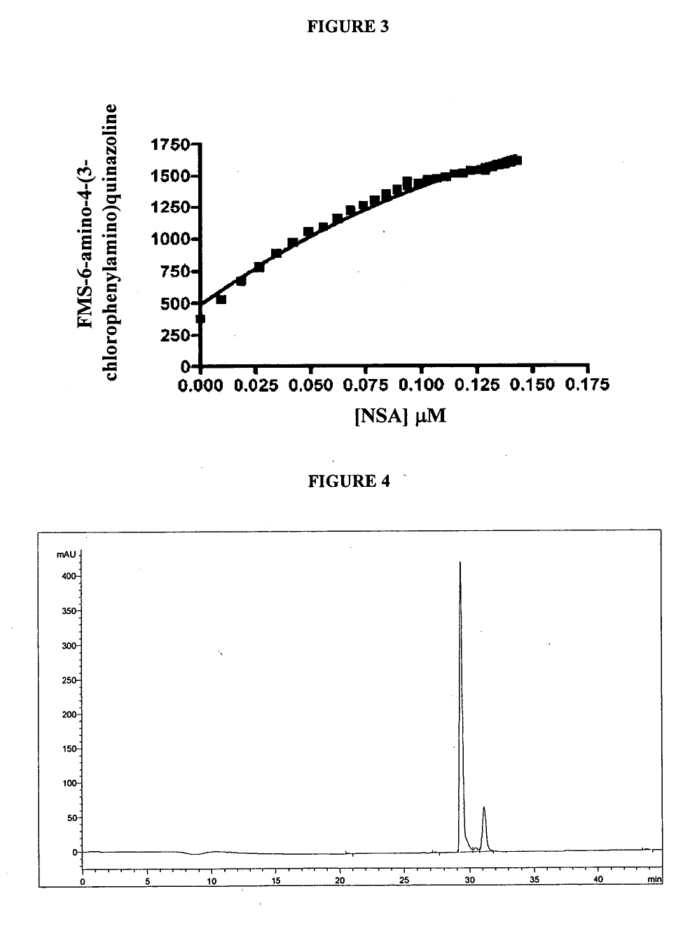

The standard protocol was then used to determine and compare the K_{d} values of 6-amino-4-(3-chlorophenylamino)quinazoline and FMS-6-amino-4-(3-chlorophenylamino)quinazoline. The results of the fluorescence titrations for each sample (as a function of added NSA) are depicted graphically in FIGS. 1 to 3.

Hydrolysis of FMS Derivative in Mouse Plasma

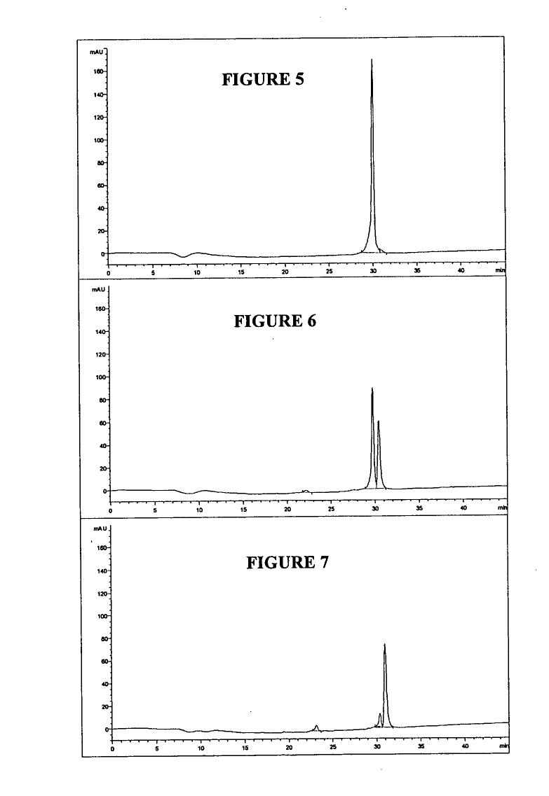

Mouse plasma was incubated with 100 μM FMS-6-amino-4-(3-chlorophenylamino)quinazoline at 37° C. Samples were taken and analysed on RP-HPLC as described above in Example 1b) ii). The relative amounts of the two peaks were calculated. Chromatograms of samples taken at 0 (see FIG. 5), 5 (see FIG. 6) and 22 (see FIG. 7) hours show the relative change in the amount of the two compounds (i.e. FMS-6-amino-4-(3-chlorophenylamino)quinazoline vs 6-amino-4-(3-chlorophenylamino)quinazoline) in the plasma after incubation at 37° C. FMS-6-amino-4-(3-chlorophenylamino)quinazoline is almost completely hydrolysed to 6-amino-4-(3-chlorophenylamino)quinazoline in mouse plasma at 37° C. after 28 hours.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A compound of formula (I) 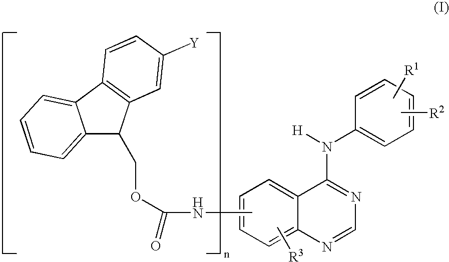
R¹ and R² are independently selected from hydrogen, hydroxy, halogen, nitro, cyano, optionally substituted alkyl, optionally substituted alkenyl, optionally substituted alkynyl, optionally substituted alkoxy, optionally substituted aryloxy, optionally substituted arylalkyloxy and optionally substituted heteroarylthio;
R³ is selected from hydrogen, hydroxy, halogen, nitro, cyano, carboxy, optionally substituted alkyl, optionally substituted alkoxy, aminoacyl, optionally substituted heteroaryl, and NR⁴R⁵ wherein R⁴ and R⁵ are independently selected from hydrogen, alkyl, cycloalkyl, aryl, —C(O)optionally substituted alkenyl, —C(O)optionally substituted alkyl and —C(O)optionally substituted alkynyl, or where R⁴ and R⁵ together with the N atom represent a 5, 6 or 7 membered nitrogen containing heterocycle;
Y is selected from hydrogen or sulfonic acid (SO₃H); and
n is either 1 or 2.

2. A compound according to claim 1 wherein R¹ and R² are independently selected from hydrogen, hydroxy, halogen, nitro, cyano, C₁-C₄ alkyl, C₁-C₄ halo substituted alkyl, optionally substituted C₂-C₄ alkenyl, C₂-C₄ alkynyl, C₁-C₄ alkoxy, C₁-C₄ halo substituted alkoxy, halo substituted aryloxy, halo substituted arylalkyloxy, aryloxy, arylalkyloxy, and substituted heterarylthio.

3. A compound according to claim 1 wherein R¹and R² are independently selected from hydrogen, hydroxy, fluoro, chloro, bromo, nitro, cyano, methyl, ethyl, 1-chloroethyl, trifluoromethyl, vinyl, nitrovinyl, cyanovinyl, trifluorovinyl, 1-propynyl, ethynyl, methoxy, ethoxy, 1-chloroethyloxy, phenoxy, benzyloxy, 3-F-benzyloxy, substituted thioimidazole.

4. A compound according to claim 1 wherein R¹and R² are independently selected from hydrogen, fluoro, chloro, bromo, methyl, trifluoromethyl, vinyl, ethynyl, phenoxy, benzyloxy, 3-F-benzyloxy and 1-methylimidazol-2-ylthio.

5. A compound according to claim 1 wherein R₁ and R² represent the following pairs of substituents: 3-Cl, 4-H; 3-Br, 4-H; 3-methyl, 4-H; 3-Cl, 4-F; 3-ethynyl, 4-H; 3-H, 4-H; 3-Cl, 4-OCH₂(3-FC₆H₄); 3-H, 4-OC₆H₅; 3-Br, 4-(1-methylimidazol-2-ylthio); and 3-H, 4-OCH₂C₆H₅.

6. A compound according to claim 1 wherein R³ is selected from hydrogen, hydroxy, halogen, nitro, cyano, C₁-C₄ alkyl, C₁-C₄ halo substituted alkyl, C₁-C₄ alkoxy, C₁-C₄ substituted alkoxy, optionally substituted heteroaryl, aminoacyl, and NR⁴R5 where R⁴ and R⁵ independently represent alkyl, —C(O)optionally substituted alkyl, —C(O)optionally substituted alkenyl, —C(O)optionally substituted alkynyl, or R⁴ and R⁵ together with the N atom represent morpholinyl or piperidinyl.

7. A compound according to claim 1 wherein R³ is selected from hydrogen, hydroxy, fluoro, chloro, bromo, nitro, cyano, methyl, ethyl, 1-chloroethyl, methoxy, ethoxy, 1-chloroethoxy, methoxyethoxy, 2-(N-morpholinyl)ethoxy, 3-(N-morpholinyl)propoxy, carbamoyl, N-(C₁-C₄)alkyl carbamoyl, N,N′-di-(C₁-C₄)alkyl carbamoyl, C₁-C₄ mono or dialkylamino, —NHC(O)CH₂SSCH₃, NHC(O)CHCHCH₂(3-ethoxy-N-morpholinyl), acrylamidyl, butynamidyl, propanamidyl, morpholinyl, piperidinyl, imidazolyl, 2-((2-methanesulphonyl-ethylamino)methyl)-furan-2-yl and 5-morphalinomethylthien-3-yl.

8. A compound according to claim 1 wherein R³ is selected from hydrogen, hydroxy, fluoro, chloro, methoxy, ethoxy, methyl, methoxyethoxy, 2-(N-morpholinyl)ethoxy, 3-(N-morpholinyl)propoxyl, N-methylamino, N,N′-dimethylamino, acrylamidyl, butynamidyl, propanamidyl, 1-imidazolyl, —NHC(O)CH₂SSCH₃, —NHC(O)CHCHCH₂(3-ethoxy-N-morpholinyl), morpholinyl, piperidinyl, 2-((2-methanesulphonyl-ethylamino)methyl)-furan-2-yl and 5-morpholinomethylthien-3-yl.

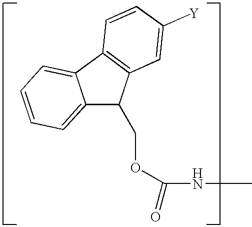

9. A compound according to claim 1 wherein the prodrug moiety
is located at the 7- and/or 6- positions of the quinazoline ring.

10. A compound according to claim 1 wherein n is 1.

11. A compound according to claim 10 wherein the prodrug moiety is located at the 6-position of the quinazoline ring.

12. A compound according to claim 11 wherein the prodrug moiety is located at the 6-position of the quinazoline ring and R³ is located at the 7-position.

13. A compound according to claim 1 wherein n is 2 and both Y are the same.

14. A compound according to claim 13 wherein the prodrug moieties are located at the 6 and 7-positions of the quinazoline ring.

15. A compound according to claim 1 selected from a compound wherein: (i) R¹ is 3-Cl, R² and R³ are H, n is 1 and Y is SO₃H; or (ii) R¹ is 3-Cl, R², R³ and Y are H and n is 1.

16. A compound according to claim 15 wherein the prodrug moiety is located at the 6-position.

17. A pharmaceutical composition comprising a therapeutically effective amount of at least one compound of formula (I) according to claim 1 or a pharmaceutically acceptable salt thereof as an active ingredient.

18. A method for preparing a compound of formula (I) as defined in claim 1 comprising contacting a compound of formula (II): 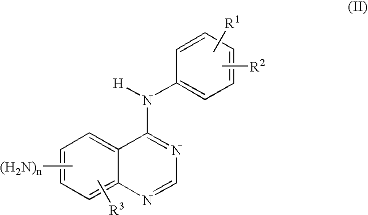
(wherein R¹-R³ and n are as defined in claim 1),
with 9-fluorenylmethoxycarbonylchloride (FM-Cl),
9-fluorenylmethoxycarbonylchloride-2-sulfonic acid (FMS-Cl), 9-fluorenylmethoxycarbonyloxysuccinimide (FMOC-OSuc) or 9-fluoroenylmethoxycarbonyl-N-hydroxysuccinimide-2-sulfonic acid (FMS-OSuc), for a time and under conditions sufficient to form said compound of formula (I), and optionally performing a chemical modification.

19. A method according to claim 18 wherein the compound of formula (II) is contacted with FMS-Cl and a base in a solvent and is stirred at room temperature.

20. A method according to claim 19 wherein the base is potassium acetate and the solvent is ethyl acetate.

21. A method according to claim 18 wherein the compound of formula (II) is contacted with FMOC-OSuc in a solvent and stirred at room temperature.

22. A method according to claim 21 wherein the solvent is dichloromethane.

23. A method for the treatment or prevention of proliferative disorders including the step of administering to a subject a therapeutically effective amount of a compound of formula (I): 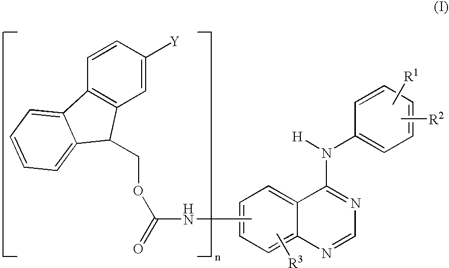
wherein:
R¹ and R² are independently selected from hydrogen, hydroxy, halogen, nitro, cyano, optionally substituted alkyl, optionally substituted alkenyl, optionally substituted alkynyl, optionally substituted alkoxy, optionally substituted aryloxy, optionally substituted arylalkyloxy and optionally substituted heteroarylthio;
R³ is selected from hydrogen, hydroxy, halogen, nitro, cyano, carboxy, optionally substituted alkyl, optionally substituted alkoxy, aminoacyl, optionally substituted heteroaryl, and NR⁴R⁵ wherein R⁴ and R⁵ are independently selected from hydrogen, alkyl, cycloalkyl, aryl, —C(O)optionally substituted alkenyl, —C(O)optionally substituted alkyl and —C(O)optionally substituted alkynyl, or where R⁴ and R⁵ together with the N atom represent a 5, 6 or 7 membered nitrogen containing heterocycle;
Y is selected from hydrogen or sulfonic acid (SO₃H); and
n is either 1 or 2.

24. A method according to claim 23 wherein the proliferative disorder is selected from human cancers, psoriasis, benign prostatic hypertrophy, atherosclerosis and restenosis.

25. A method according to claim 24 wherein the human cancers are selected from lung, bladder, oesophageal, gastrointestinal, prostate, leukaemia, ovarian, bronchial, and pancreatic cancers.
